(19) United States
(12) Patent Application Publication
(10) Pub. No.: US 2003/0113384 A1
(43) Pub. Date: Jun. 19, 2003

(54) Antimicrobial compositions for mastitis control

(75) Inventors: Dale Lind Fredell, Lindstrom, MN (US); Francis Lawrence Richter, Lino Lakes, MN (US); Benjamin R. Bode, Algona, IA (US)

Correspondence Address: MERCHANT & GOULD PC, P.O. BOX 2903, MINNEAPOLIS, MN, 55402-0903, US

(73) Assignee: Ecolab Inc., St. Paul, MN

(21) Appl. No.: 10/217,583

(22) Filed: Aug. 12, 2002

Related U.S. Application Data
(63) Continuation of application No. 09/419,251, filed on Oct. 15, 1999, now abandoned.

(51) Int. Cl. A61K 33/36; A61K 31/19

(52) U.S. Cl. 424/672; 514/557

(57) ABSTRACT

Antimicrobial compositions containing an iodine compound and a carboxylic acid, for example, a fatty acid, are disclosed. The compositions can be formulated for use as a surgical scrub, wound antiseptic, pre-operative skin preparation, industrial sanitizer, antimicrobial soap, teat dip, etc. In one particularly advantageous embodiment, a composition of the invention is formulated as a teat dip further including a rheology modifier, at least one surfactant, suitable emollients, skin conditioners and lubricants.

### FIELD OF THE INVENTION

The present invention is directed to antimicrobial compositions. In some embodiments, the invention provides compositions and formulations for the control of mastitis in milk producing animals. In one preferred embodiment, the invention provides teat dip formulations comprising an iodine compound and a carboxylic acid, typically a fatty acid.

### BACKGROUND OF THE INVENTION

Mastitis is an inflammation of the mammary gland. Bovine mastitis is the most common and most costly disease affecting dairy herds. Some estimates suggest at least half of the dairy animal population have some degree or form of mastitis. This condition results in lowered milk yield and reduced milk quality. Economic loss to mastitis in the U.S. is estimated at about $1.8 billion or approximately 10% of total milk sales with about two-thirds of this loss due to reduced milk production from infected cows. In dairy cattle, mastitis typically results from microorganisms, usually bacteria, that invade the udder, multiply in the milk producing tissues, and synthesize toxins, a by-product of bacterial metabolism. The characteristic features of inflammation are swelling, heat, redness, pain and disturbed function.

While the animal immune system can fight intramammary infections, many chronic infections remain sub-clinical (asymptomatic) and undetected unless diagnosed by laboratory testing. Sub-clinical mastitis can result in a reservoir of micro-organisms which can lead to the infection of other animals within the herd. More than 80 species of microorganisms have been identified as causal agents, although approximately 95% of mastitis is caused by four pathogens; Staphylococcus aureus, Streptococcus agalactiae, Streptococcus dysagalactiae, and Streptococcus uberis. Mastitis causing pathogens fall into two categories namely contagious and environmental. Contagious bacteria, such as Streptococcus agalactiae and Staphylococcus aureus, primarily colonize host tissue sites such as mammary glands, teat canals, teat skin lesions etc. and are spread from one infected cow to another during the milking process. Environmental bacteria, often streptococci, enterococci and coliform organisms, are commonly present within the cow's surroundings from sources such as cow feces, soil, plant material, bedding or water, and infect by casual opportunistic contact with an animal. This distinction, although not exclusive, is of practical importance because different dairy herd maintenance measures are needed for the different groups of microorganisms. In all bovine mastitis cases, whatever the causal microorganism, the route of transmission of the invading pathogen into the inner gland of the udder is through the teat orifice and teat canal.

Management of dairy herds focuses attention on treatment of both established mastitis and on prevention of new intramammary infections. Therapy and hygiene are two fundamental components of an effective mastitis control program. Each is applied in concert, and each operates independently. The primary effect of therapy is to eliminate established infections; whereas, hygiene reduces the incidence of new infection by interrupting transmission vectors. A non-exhaustive list of ancillary factors which may be employed for the elimination and prevention of mastitis, include, post-lactation antibiotic infusion into the udder (e.g.,. dry cow treatment); and, post-milking teat antisepsis or “teat dipping” during lactation.

Researchers agree, and an abundance of published evidence supports the concept, that dipping teats into an effective antimicrobial solution immediately after each milking is the single most effective procedure for decreasing new intramammary infections in lactating cows. Between 1955 to 1970, Dodd and co-workers conducted extensive epidemiologic investigations in commercial dairy herds (F. K. Neave, F. H. Dodd, and R. G. Kingwell, 1966, “A Method of Controlling Udder Disease”, Vet. Rec. 78:521; F. K. Neave, F. H. Dodd, R. G. Kingwell and D. R. Westgarth, 1969, “Control of Mastitis in the Dairy Herd by Hygiene and Management”, J. Dairy Sci. 52:696; F. H. Dodd, D. R. Westgarth, F. K. Neave and R. G. Kingwill, 1969, “Mastitis—The Strategy of Control”, J. Dairy Sci. 52:689; and F. H. Dodd, and F. K. Neave, 1970, “Mastitis Control”, Proceedings, Nat'l. Inst. Res. Dairying, pp. 21-60). From this work, they developed the conceptual basis for modem mastitis control methods of which teat dipping is an integral component. The efficacy and value of teat dipping has since been confirmed in dozens of field trials, and it is now accepted that an effective teat dip can reduce the incidence of new intramammary infections at least 50% and often up to 90%.

To reduce mastitis, commercial teat dips have been developed containing a variety of antimicrobial agents including iodophors, quaternary ammonium compounds, chlorhexidine salts, chlorine release compounds (e.g. alkali hypochlorites), oxidizing compounds (e.g. hydrogen peroxide, peracids), protonated carboxylic acids (e.g. heptanoic, octanoic, nonanoic, decanoic, undecanoic acids), acid anionics (e.g. alkylaryl sulfonic acids), and chlorine dioxide (from chlorite). These agents, which have varying degrees of effectiveness, limit the transmission of mastitis by reducing pathogen populations on the teat. Teat dips, can also be divided into two broad classifications. The Class I type are antimicrobial and are applied to kill microorganisms already present in the teat canal or on the surface of the teat skin. By design, their microbiological effect is immediate and they primarily target the contagious organisms that are vectored between animals during the pre-milking, milking and post-milking process. The Class II type teat dip, often referred to as a “teat sealer,” is a film-forming or coating composition which may or may not be antimicrobial; and, functions by developing a residual protective barrier on the teat thus providing prophylaxis by sealing the teat orifice from environmental contamination. The film which forms on the surface of the teat serves as a physical barrier through which mastitis causing pathogens cannot penetrate during the intermilking period.

General disclosures of teat dip technology are shown in, for example, “Current Concepts of Bovine Mastitis.” 1996, Fourth Ed. National Mastitis Council, Madison Wis.; P. A. Murdough and J. W. Pankey, 1993. “Evaluation of 57 Teat Sanitizers Using Excised Cow Teats”, J. Dairy Sci. 76:2033-2038; J. W. Pankey et al., 1984, “Uptake on Post-milking Teat Antiseptics”, J. Dairy Sci. 67:1336-1353; R. J. Farnsworth, 1980, “Role of Teat Dips in Mastitis Control”, J. Am. Vet. Med. Assoc. 76:1116-1118; W. N. Philpot, 1979, “Control of Mastitis by Hygiene and Therapy”, J. Dairy Sci. 62:168-176; W. N. Philpot and J. W. Pankey, 1978, “Hygiene in the Prevention of Udder Infections V. Efficacy of Teat Dips Under Experimental Exposure to Mastitis Pathogens”, J. Dairy Sci. 61:956-963; R. P. Natzke, 1977, “Role of Teat Dips and Hygiene is Mastitis Control”, J. Amer. Vet. Med. Assoc. 170:1196-1198; W. N. Philpot and J. W. Pankey, 1975, “Hygiene in the Prevention of Udder Infections. III. Effectiveness of 59 Teat Dips for Reducing Bacterial Populations on Teat Skin”, J. Dairy Sci. 58:209-216; R. J. Eberhart and J. M. Buckalew, 1972, “Evaluation of a Hygiene and Dry Period Therapy Program for Mastitis Control”, J. Dairy Sci. 55:1683-1691; W. D. Schultze and J. W. Smith, 1972, “Effectiveness of Postmilking Teat Dips”, J. Dairy Sci. 55:426-431; D. P. Wesen and L. H. Schultz, 1970, “Effectiveness of a Post-Milking Teat Dip in Preventing New Udder Infections”, J. Dairy Sci. 53:1391-1403; and British Pat. No. 1,144,637 (Kelco Chemicals Ltd.), published Mar. 5, 1969.

Typical disclosures of intermilking or protective (barrier-type) film-forming teat dips or teat “sealers” can be found in Akers et. al., U.S. Pat. No. 3,066,071, issued Nov. 27, 1962; Kraus, U.S. Pat. No. 3,222,252, issued Dec. 7, 1965 (but, see Philpot et. al., J. Dairy Science 58:205-216); Coughman and Brown, U.S. Pat. No. 3,993,777, issued Nov. 23, 1976; Pugliese, U.S. Pat. No. 4,049,830, issued Sep. 20, 1977; and Andrews et al., U.S. Pat. No. 4,113,854, issued Sep. 12, 1978. One disadvantage of many such film-forming agents is their tendency to form a “hard” film which is tenacious and often difficult to remove.

Thus, although many teat dip products are available, there is a continuing need for new and effective teat dip compositions having immediate and long lasting antimicrobial effect against a wide spectrum of mastitis causing organisms. Moreover, there is a continuing need for new and effective antimicrobial compositions for medical or hygiene purposes.

### SUMMARY OF THE INVENTION

The present invention is directed to novel antimicrobial compositions which can be used as a surgical scrub, wound antiseptic, pre-operative skin preparation, industrial sanitizer, antimicrobial soap, etc. In one presently preferred embodiment, an antimicrobial composition of the invention can be formulated for use as a teat dip for milk producing animals.

It will be noted that at several places throughout the Specification, guidance is provided through lists of examples. In each instance, the recited lists serve only as representative groups. It is not meant, however, that the lists are exclusive.

In general, an antimicrobial composition of the invention comprises an iodine compound, a C₆-C₁₂ fatty acid and a carrier. In preferred embodiments, the fatty acid can be a C₇-C₉ fatty acid. A particularly preferred fatty acid is heptanoic acid. The iodine compound can be in the form of an iodophor. In some embodiments, the iodine component of the iodophor can be provided as an iodine premix, for example, NaI/I₂.

In a working composition of the invention, the ratio of fatty acid:titratable iodine can be about 1:25 to 3.3:1, in some embodiments about 1:3 to 1:2 and in some preferred embodiments, about 1:2 to 1:1.

In preferred embodiments of a teat dip composition, the teat dip formulation also includes one or more surfactants. In some embodiments, the compositions include at least one surfactant and an iodine compound, such as NaI/I₂, in a ratio of total surfactant:titratable I₂ of about 4:1 to 13:1, and preferably about 9:1-11:1. A teat dip composition can also include a rheology modifier, a film-forming agent or admixture, a buffer system, a hydrotope, a coupler, an emollient, skin conditioner or a lubricant or admixtures thereof.

### DETAILED DESCRIPTION OF THE INVENTION

The present invention is directed to antimicrobial compositions. In general, the compositions include an iodine compound and a fatty acid, typically a C₆-C₁₂ fatty acid. In some embodiments, the compositions of the invention can be advantageously used as a teat dip for control of mastitis in milk producing animals. It will also be appreciated, however, that the herein disclosed compositions can also be formulated as a surgical scrub, wound antiseptic, pre-operative skin preparation, industrial sanitizer, antimicrobial soap, etc.

Components Used in a Teat Dip of the Invention

The compositions of the invention can be formulated as a teat dip for mastitis prevention or control. According to this embodiment, the compositions can comprise an iodine compound and a carboxylic acid, such as a fatty acid in a carrier. Teat dip compositions may optionally also include, a rheology modifier or admixture, a film-forming agent or admixture, a buffer system, a hydrotrope, a coupler or admixtures thereof, a surfactant or surfactant admixture, an emollient, a skin-conditioner or a lubricant or admixtures thereof, one or more adjuvants, etc. The preferred compositions of the invention comprise ingredients which are generally regarded as safe, and are not of themselves or in admixture incompatible with milk or milk by-products. Ingredients may also be selected which are cooperative in their combined effects whether incorporated for physical integrity of the formulation or to facilitate healing and overall health of the teat. The carrier functions to dilute the active ingredients and facilitate application to the intended surface.

The compositions of the invention can be provided as a ready-to-use formulation or as concentrates which are diluted prior to use. Hence, throughout this disclosure, reference will be made to “working compositions” which are the compositions which are actually used as a teat dip, surgical scrub, pre-operative skin preparation etc. Thus, a working composition includes, for example, ready-to-use compositions as well as concentrates which have been diluted for use in a particular application. Methods for preparing concentrates based on the disclosure herein of working compositions are within the knowledge of one of skill in the art.

In preferred embodiments, a teat dip composition of the invention provides a soft barrier over the teat. A “soft barrier” provides a self annealing barrier which can flow to re-cover areas of the teat from which the dip may have been removed when the animal lays down, walks through the pasture or is subject to some other event which causes inadvertent removal of the dip. Advantageously, however, a herein disclosed soft barrier can also be readily removed from the teat using routine washing procedures prior to milking without congealing, pilling or leaving some other undesired residue on the teat. The soft barrier is provided by surfactants and rheology modifiers described further below. Thus, unlike prior film-forming barriers, the soft barrier provided herein is a soft, non-peeling barrier that can undergo plastic deformation, for self-repair, without breaking or cracking.

Carrier

The carrier of a composition of the invention can generally be an aqueous medium such as water, or an organic liquid such as an oil, a surfactant, an alcohol or polyol, an ester, an ether, or an organic or aqueous mixture of any of these. Water is preferred as a carrier or diluent in compositions of the invention. However, in some embodiments the carrier can include low concentrations of short chain alcohols (i.e., <5%) or other inert aqueous soluble liquids.

Iodine Compound

The iodine compounds of the invention provide a portion of the antimicrobial activity of the compositions of the invention and are selected to provide disinfecting or sanitizing antimicrobial efficacy within the definitions of the Association of Analytical Chemists Official Methods of Analysis §960.09 entitled “Germicidal and Detergent Sanitizing Action of Disinfectants.” Iodine compounds suitable for a composition of the invention are known and disclosed in, for example, U.S. Pat. Nos. 4,271,149; 5,310,549; 5,368,868; and 5,503,838, the entire disclosures of which are incorporated herein by reference.

In preferred embodiments, the iodine compound can be present in the form of an iodophor. That is, the iodine compound is present as a complex with one or more suitable surfactants, or other suitable complexing compound, such as, for example, polyvinyl pyrrolidone (PVP). Typically, the iodophor can be present in a composition of the invention to provide approximately 0.5 ppm to 6.0 ppm, preferably about 1.0 ppm to 3.0 ppm, of free iodine in the working composition. Iodophors are known and disclosed in, for example, U.S. Pat. Nos. 5,618,841; 5,310,549; etc. The entire disclosure of each of these patents are incorporated herein by reference. Preferred surfactants suitable for preparing an iodophor with one or more surfactants are further discussed below.

In some embodiments, the iodine can be provided as an iodine premix, for example, NaI/I₂, to form an iodophor suitable for the invention. The iodine premix can be formulated by adding deionized water, iodine and an iodide constituent to a reactor with adequate mixing. Generally, the iodide constituent can be any alkaline earth metal-iodine salt such as sodium iodide or potassium iodide. According to this embodiment, a working composition preferably provides about 0.1% to about 1.5%, typically about 1.0% of titratable I₂. An NaI/I₂ premix at a concentration of about 1.8% of the total weight of the working composition provides about 1% titratable I₂ in the working composition. Some such compositions are further described in the Examples.

Surgical scrubs according to the invention preferably provide about 0.5% titratable I₂ and pre-operative skin preparations about 1% titratable I₂.

Fatty Acid

The compositions of the invention also include a carboxylic fatty acid component which provides a portion of the antimicrobial activity. For optimal antimicrobial activity, the pH of the composition is preferably at or below the pKa of the fatty acid. Thus, in preferred compositions, the relationship of pH to pKa provides antimicrobial activity through carboxylic fatty acids which are substantially protonated. Preferred compositions of the invention have a pH in the range of about 3.5 to about 6.0, typically about 4.0 to about 5.0 and, in one preferred embodiment, about 4.2. Although a wider range of pH is possible, typically, below pH 3.5 undesirable skin irritation may occur and above pH 6.0 dissociation or conversion to the ionized form may reduce the antimicrobial efficacy of the fatty acid.

Fatty acids suitable for a composition of the present invention include C₆-C₁₂ fatty acids. Preferred fatty acids have a chain length from about C₇-C₉. One particularly preferred fatty acid is heptanoic acid which has seven carbon atoms, including the carboxyl group, and has a pKa of 4.4. In addition to its preferred water solubility, heptanoic acid is not significantly irritating to the tissues.

In general, the fatty acid component of the invention can be present at about 0.01% to about 5.0% of the total weight of the working composition. For example, when heptanoic acid is the fatty acid, it may be present at about 0.01% to about 5% of the total weight of the working composition. In a working composition having a pH of about 4.2, heptanoic acid may be present at about 0.1% to about 5% of the total working solution, preferably at about 0.5% to about 1.5%.

The antimicrobial components of the composition can be mixed in the carrier and include buffers, surface active agents and/or couplers to provide a pH and solubility suitable for efficient bactericidal effect with low or no irritation to the tissues of the teat. The buffer system is present to prevent the likelihood of pH drift under typical use conditions. In general, the buffer system can include any weak acid and its conjugate base. Preferred bases used to adjust the pH of the compositions include hydroxides of the alkaline earth metals, for example NaOH, KOH, LiOH, etc.

Maintenance of the pH of compositions described in this invention is preferred to minimize undesirable chemical changes which may inhibit the microbiological efficacy of the antimicrobial components or cause toxic or irritating effect upon the teat. Any compatible organic or inorganic material or mixture of materials which has the desired effect of maintaining the composition pH within prescribed ranges can be utilized as the buffering agent or system in the invention. Factors which may cause undesirable pH shifts include the presence of naturally occurring chemicals brought into the composition, after application onto the teat, by skin exudations, milk or environmental soils; and, pH drifting which sometimes accompanies chemical equilibriums established within compositions as ingredients are changed or concentrations varied, for example, concentration changes which can occur as a teat dip dries on the teat.

In general, the pH of bovine mastitis control treatments can vary from a low of about pH 2.5 to a maximum of approximately 10.5 depending primarily upon the choice of antimicrobial agent being incorporated in the composition. Therefore the buffering agent or system is chosen accordingly. Most common commercially-available weak inorganic and organic acids can be used in the invention. Preferred weak inorganic acids include phosphoric acid and sulfamic acid. Useful weak organic acids include acetic acid, hydroxyacetic acid, citric acid, tartaric acid, lactic acid, glycolic acid, adipic acid, succinic acid, propionic acid, malic acid, alkane sulfonic acids, cycloalkane sulfonic acids, etc. Mixtures of organic and inorganic acids can also be used. One typical and preferred buffer system is citric acid and its alkali metal salt.

Solubilizing agents called hydrotropes or couplers may be generally used in compositions of the invention to maintain physical single phase integrity and storage stability. To this end, any number of ingredients known to those skilled in the formulation art may be employed, such as monofunctional and polyfunctional alcohols. These preferably contain from about 1 to about 6 carbon atoms and from 1 to about 6 hydroxy groups. Examples include ethanol, isopropanol, n-propanol, 1,2-propanediol, 1,2-butanediol, 2-methyl-2,4-pentanediol, mannitol and glucose. Also useful are the higher glycols, polyglycols, polyoxides, glycol ethers and propylene glycol ethers. Additional useful hydrotropes include the free acids and alkali metal salts of sulfonated alkylaryls such as toluene, xylene, cumene and phenol or phenol ether or diphenyl ether sulfonates; alkyl and dialkyl naphthalene sulfonates and alkoxylated derivatives. One preferred hydrotrope is 1-octane sulfonate and 1,2-octane disulfonate.

Additional Components

A composition of the invention may also contain one or more rheology modifiers, to enhance viscosity, or thicken the composition to facilitate adherence of a dip to the teat. Adherence enables the composition to remain in contact with transient and resident pathogenic bacteria for longer periods of time, promoting microbiological efficacy and resisting waste because of excessive dripping. The rheology modifier may be a film former or act cooperatively with a film-forming agent to form a barrier that provides additional protection. However, in preferred embodiments, a teat dip composition of the invention provides a soft barrier, rather than a film.

Water soluble or water dispersible rheology modifiers that are useful can be classified as inorganic or organic. The organic thickeners can further be divided into natural and synthetic polymers with the latter still further subdivided into synthetic natural-based and synthetic petroleum-based.

Inorganic thickeners are generally compounds such as colloidal magnesium aluminum silicate (VEEGUM®), colloidal clays (Bentonites), or silicas (CAB-O-SILS®) which have been fumed or precipitated to create particles with large surface to size ratios. Suitable natural hydrogel thickeners are primarily vegetable derived exudates. For example, tragacanth, karaya, and acacia gums; and extractives such as caragheenan, locust bean gum, guar gum and pectin; or, pure culture fermentation products such as xanthan gum. Chemically, all of these materials are salts of complex anionic polysaccharides. Synthetic natural-based thickeners having application are cellulosic derivatives wherein the free hydroxyl groups on the linear anhydro-glucose polymers have been etherified or esterified to give a family of substances which dissolve in water and give viscous solutions. This group of materials includes the alkyl and hydroxyllalkycelluloses, specifically methylcellulose, hydroxyethylmethylcellulose, hydroxypropylmethylcellulose, hydroxybutylmethycellulose, hydroxyethylcellulose, ethylhydroxyethylcellulose, hydroxypropylcellulose, and carboxymethylcellulose. Synthetic petroleum-based water soluble polymers are prepared by direct polymerization of suitable monomers of which polyvinylpyrrolidone, polyvinylmethylether, polyacrylic acid and polymethacrylic acid, polyacrylamide, polyethylene oxide, and polyethyleneimine are representative.

A preferred rheology odifier is xanthan gum, for example, KELZAN™T available manufactured by Kelco Biopolymer. This rheology modifier is particularly advantageous in that it is a pseudoelastic composition having non-thixotropic properties.

Suitable surfactants or surfactant admixtures for forming an iodophor can be selected from compatible water soluble or water dispersible nonionic, or anionic surface-active agents; or mixtures of each or both types. In preferred embodiments, the surfactant is a non-ionic surfactant. Non-ionic surfactants useful in the invention are generally characterized by the presence of an organic hydrophobic group and an organic hydrophilic group and are typically produced by the condensation of an organic aliphatic, alkyl aromatic or polyoxyalkylene hydrophobic compound with a hydrophilic alkaline oxide moiety which in common practice is ethylene oxide or a-polyhydration product thereof, polyethylene glycol. Practically any hydrophobic compound having a hydroxyl, carboxyl, amino, or amido group with a reactive hydrogen atom can be condensed with ethylene oxide, or its polydration adducts, or its mixtures with alkoxylenes such as propylene oxide to form a nonionic surface-active agent. The length of the hydrophilic polyoxyalkylene moiety which is condensed with any particular hydrophobic compound can be readily adjusted to yield a water dispersible or water soluble compound having the desired degree of balance between hydrophilic and hydrophobic properties.

Useful nonionic surfactants in the present invention include: Block polyoxypropylene-polyoxyethylene polymeric compounds based upon propylene glycol, ethylene glycol, glycerol and trimethylolpropane as the initiator reactive hydrogen compound. Examples of polymeric compounds made from a sequential propoxylation and ethoxylation of initiator are commercially available under the trade name PLURONIC® manufactured by BASF Corp. PLURONIC® compounds are difunctional (two reactive hydrogens) compounds formed by condensing ethylene oxide with a hydrophobic base formed by the addition of propylene oxide to two hydroxyl groups of propylene glycol. This hydrophobic portion of the molecule weighs from about 1,000 to about 4,000. Ethylene oxide is then added to sandwich this hydrophobe between hydrophilic groups, controlled by length to constitute from about 10% by weight to about 80% by weight of the final molecule.

Likewise useful nonionic surfactants include condensation products of one mole of a saturated or unsaturated, straight or branched chain alcohol having from about 6 to about 24 carbon atoms with from about 3 to about 50 moles of ethylene oxide. The alcohol moiety can consist of mixtures of alcohols in the above delineated carbon range or it can consist of an alcohol having a specific number of carbon atoms within this range. Examples of like commercial surfactant are available under the trade name NEODOL® manufactured by Shell Chemical Co. and ALFONIC® manufactured by Vista Chemical Co.

Condensation products of one mole of saturated or unsaturated, straight or branched chain carboxylic acid having from about 8 to about 18 carbon atoms with from about 6 to about 50 moles of ethylene oxide. The acid moiety can consist of mixtures of acids in the above delineated carbon atoms range or it can consist of an acid having a specific number of carbon atoms within the range. Examples of commercial compounds of this chemistry are available on the market under the trade name NOPALCOL® manufactured by Henkel Corporation and LIPOPEG® manufactured by Lipo Chemicals, Inc. In addition to ethoxylated carboxylic acids, commonly called polyethylene glycol esters, other alkanoic acid esters formed by reaction with glycerides, glycerin, and polyhydric (saccharide or sorbitan/sorbitol) alcohols have application in this invention. All of these ester moieties have one or more reactive hydrogen sites on their molecule which can undergo further acylation or ethylene oxide (alkoxide) addition to control the hydrophilicity of these substances.

Also useful nonionic surfactants include the condensation products of one mole of alkyl phenol wherein the alkyl constituent contains from about 8 to about 18 carbon atoms with from about 3 to about 50 moles of ethylene oxide. The alkyl group can, for example, be represented by diisobutylene, di-amyl, polymerized propylene, isoctyl, nonyl, and di-nonyl. Examples of commercial compounds of this chemistry are available on the market under the trade name IGEPAL® manufactured by Rhone-Poulenc and TRITON® manufactured by Union Carbide. The surfactants used in the present compositions are also selected to improve solubility for removal of the composition from the teat prior to milking. A presently preferred non-ionic surfactant for improving water solubility is nonylphenol ethoxylate 12 mole (NPE 12), for example, IGEPAL® CO-720 available from Rhone-Poulenc.

In general, a composition of the invention preferably provides a ratio of total surfactant:titratable I₂ from about 4:1 to 13:1, typically about 6:1 to 12:1 and in some preferred embodiments, about 9:1-11:1.

Teat dip compositions of the present invention can also include an emollient and/or humectant to lubricate, condition and generally reduce and promote the healing of irritation of the teat surface of which may result either from the antimicrobial components, from the mechanical action of the milking machine or from environmental conditions such as wind chill, dehydration, abrasion and sunburn. Any water soluble or dispersible skin conditioning agent may be used in this invention. Compositions such as polyhydric alcohols are useful in the invention including glycerin, sorbitol, mannitol, and propylene glycol and its homopolymers; fatty acid esters of simple monohydril alcohols including isopropyl palmitate or isopropyl myristate and similar esters; polyol esters of fatty acids; and, ethoxylated lanolins, vegetable oils, and similar natural sourced derivatives such as aloe. Preferred emollients to be used in the invention include glycerin, and propylene glycol.

The compositions of the invention may also optionally include medicaments, for example sunscreens such as paraamino benzoic acid and healing agents such as allantoin or urea to provide curative action and stimulation of formation of new tissue; preservatives such as methyl paraben, propyl paraben, sorbic and benzoic acids or salts thereof to retard bacterial growth and prolong shelf life; antioxidants such as BHT (butylated hydroxytoluene), BHA (butylated hydroxyanisole), TBHQ (tert-butylhydroquinone), or propyl gallate to retard oxidative or hydrolytic degradation; sequestering agents such as aminopolyacetates, polyphosphonates, aminopolyphosphonates, polycarboxylates, and condensed phosphates; dispersants or suspending agents having polyelectrolytic character such as polyacrylate and similar polycarboxylates of homopolymeric or copolymeric structure; and manufacturing processing agents, for example defoam additives employed to facilitate blending and mixing.

The following examples are provided to further describe certain advantageous compositions according to the invention. The Examples, however, are not intended to limit the scope of the compositions within the spirit and scope of the invention.

### EXAMPLES

### Example 1

### Formulation of a Teat Dip Composition

The present Example provides one procedure for preparing a working composition of a teat dip composition according to the invention. This procedure can be used regardless of the total weight of the composition formulated. Thus, while a particular weight percentage of a component may vary among formulations, the procedure used for mixing the components is the same. It will be appreciated that other procedures can be used and are within the knowledge of one skilled in the art.

Deionized water is added to a stainless steel tank having a variable speed pitched blade turbine. The tank is agitated and a base, such as liquid KOH (45%) is added and mixed for about 5 minutes. Xanthan gum (e.g., KELZAN™T available from Kelco Biopolymers) is charged with water through an eductor funnel and mixed for approximately 1 hour or until the gum is completely solubilized. Preferably, the gum is completely solubilized prior to addition of subsequent components.

Glycerine (e.g., 96% USP), propylene glycol (technical) the fatty acid (e.g., heptanoic acid) are added and mixed for about 5 minutes to incorporate them into the mixture. A buffer, such as 50% white powdered citric acid is slowly added to the mixture and mixed for about 10 minutes. The surfactants are then added, preferably one at a time with about 15 minutes of mixing after each surfactant is added to disperse the surfactant throughout the mixture. The iodine compound is then added (e.g. NaI/I₂ premix) and the composition is mixed for 30 minutes or until the mixture appearance is uniform.

The preferred pH for a teat dip composition is about 3.5 to about 6. If the pH is less than the preferred range, a base such as potassium hydroxide can be added incrementally until the appropriate pH is achieved. If the pH is greater than the preferred range, an acid such as phosphoric acid can be added incrementally until the appropriate pH is obtained.

Table 1 provides exemplary formulations for a teat dip composition according to the invention.

TABLE 1
Teat Dip Formulations

| | A | B | C | D | E | F | G | H | I | J | K | L | M |
|---|---|---|---|---|---|---|---|---|---|---|---|---|---|
| | Wt % | Wt % | Wt % | Wt % | Wt % | Wt % | Wt % | Wt % | Wt % | Wt % | Wt % | Wt % | Wt % |
| Dionized Water | 74.40 | 74.00 | 70.90 | 70.90 | 71.90 | 71.90 | 71.90 | 72.90 | 72.90 | 70.90 | 71.90 | 71.90 | 70.90 |
| KOH 45% | 1.0 | 1.00 | 1.00 | 1.00 | 1.00 | 1.00 | 1.00 | 1.00 | 1.00 | 1.00 | 1.00 | 1.00 | 1.00 |
| Kelzan T | 0.30 | 0.30 | 0.30 | 0.30 | 0.30 | 0.30 | 0.30 | 0.30 | 0.30 | 0.30 | 0.30 | 0.30 | 0.30 |
| Glycerine 96% | 5.50 | 5.50 | 5.50 | 5.50 | 5.50 | 5.50 | 5.50 | 5.50 | 5.50 | 5.50 | 5.50 | 5.50 | 5.50 |
| Propylene Glycol | 6.00 | 6.00 | 6.00 | 6.00 | 6.00 | 6.00 | 6.00 | 6.00 | 6.00 | 6.00 | 6.00 | 6.00 | 6.00 |
| Heptanoic Acid | 0.10 | 0.50 | 1.00 | 1.00 | 1.00 | 1.00 | 1.00 | 1.00 | 1.00 | 1.00 | 1.00 | 1.00 | 1.00 |
| Citric Acid | 1.90 | 1.90 | 2.50 | 2.50 | 2.50 | 2.50 | 2.50 | 2.50 | 2.50 | 2.50 | 2.50 | 2.50 | 2.50 |
| NPE 12 | 6.00 | 6.00 | 6.00 | 6.00 | 5.00 | 5.00 | 5.00 | 4.00 | 4.00 | 6.00 | 5.00 | 5.00 | 6.00 |
| Pluronic ™ P-105 | 3.00 | 3.00 | 4.00 | 3.00 | 5.00 | 4.00 | 3.00 | 4.00 | 3.00 | 3.00 | 4.00 | 3.00 | 5.00 |
| Pluronic ™ L-44 | 0.00 | 0.00 | 1.00 | 2.00 | 0.00 | 1.00 | 2.00 | 1.00 | 2.00 | 0.00 | 0.00 | 0.00 | 0.00 |
| Pluronic ™ L-61 | 0.00 | 0.00 | 0.00 | 0.00 | 0.00 | 0.00 | 0.00 | 0.00 | 0.00 | 2.00 | 1.00 | 2.00 | 0.00 |
| NaI/I₂ Premix | 1.80 | 1.80 | 1.80 | 1.80 | 1.80 | 1.80 | 1.80 | 1.80 | 1.80 | 1.80 | 1.80 | 1.80 | 1.80 |
| | 100.00 | 100.00 | 100.00 | 100.00 | 100.00 | 100.00 | 100.00 | 100.00 | 100.00 | 100.00 | 100.00 | 100.00 | 100.00 |

All formulations were adjusted to pH 4.2 with 75% phosphoric acid.
Kelzan ™ T is a grade of xanthan gum manufactured by Kelco Biopolymers.
NPE 12 is nonylphenolethoxylate with 12 moles ethylene oxide.
Pluronic ™ P-105 is a nonionic polymeric surfactant manufactured by B.A.S.F.
Pluronic ™ L-44 is a nonionic polymeric surfactant manufactured by B.A.S.F.
Pluronic ™ L-61 is a nonionic polymeric surfactant manufactured by B.A.S.F.

### Example 2

### Simulated Teat Dip Evaluation

The “simulated teat dip evaluation” is a two part laboratory test to determine the amount of teat dip consumed, the amount retained and the amount wasted during and after application to a surface. The evaluation is performed as follows. A pre-weighed glass test tube (20 mm×150 mm) is vertically dipped to a depth of 5 centimeters from the bottom of a test tube into a pre-weighed beaker containing teat dip product. The test tube is removed from the dip and placed on a free hanging rack and allowed to drip into a clean, pre-weighed beaker (or other suitable receiving container). After dripping has ceased, the test tube is allowed to hang on a rack and dry for approximately 6 hours and reweighed.

The test tube with dried teat dip is placed vertically into a 250 ml beaker with the dip completely submersed in 200 ml ambient water with slow stirring by a magnetic rod. The time required for dissolving the dip on the test tube walls and the time required for dissolving the product bead at the bottom of the tube was recorded. The time to dissolve the dried dip on the test tube wall and the dry bead are likewise recorded (seconds). The “bead” of this test simulates the bead which forms at the end of the teat after cessation of drippage of the teat dip from the teat. The bead acts as a sealant of the teat orifice and distal teat canal.

Thus, the data recorded is total product (grams) initially placed on test tube (by weight reduction of pre-weighed beaker containing teat dip product), product lost to beaker by drippage (waste) and product retained on the test tube (the difference between total product on test tube and waste). The results of the simulated teat dip evaluation for Formulation B from Example 1 and the product BLOCKADE™(available from West-Agro, Inc.) are shown in Table 2. The free iodine concentrations were calculated using heptane partitioning.

TABLE 2
Simulated Teat Dip Evaluation

| | |
|---|---|
| BLOCKADE ® | Formula B |

| | | | | |
|---|---|---|---|---|
| Avg. Lost | 0.1854 | | 0.2645 | |
| Avg. Retain | 0.1301 | | 0.1493 | |
| Avg. Total | 0.3155 | | 0.4138 | |
| Available I₂ | — | | 1.04% | |
| Free I₂ | 3.39 | ppm | .92 | ppm |
| Dip Dissolve | 43 | sec. | 28 | sec. |
| Bead Dissolve | 54 | sec. | 43 | sec. |

### Example 3

### Sanitizing Efficacy of Teat Dip Formulations A and B Against Staphylococcus aureus With and Without a 10% Milk Challenge

Formulations A and B from Example 1 were tested for bactericidal efficacy with and without a 10% organic soil (10% sterile milk having a 2% milk fat content). The methods used for this study are those found in the Association of Analytical Chemists Official Methods of Analysis §960.09 entitled “Germicidal and Detergent Sanitizing Action of Disinfectants.”

For this Example, 1.0 ml of a culture containing 7.4×10⁷ cfu/ml Staphylococcus aureus (ATCC Deposit No. 6538) were inoculated into 99 ml of teat dip formulation as shown in Table 3 below. The S. aureus inoculum was mixed with teat dip formulation A or B with or without sterile milk.

TABLE 3
Test Formulations and Volumes

| Teat Dip | ml of Teat Dip | | Final Volume |
|---|---|---|---|
| Formulation | Formulation | ml of Milk | in ml* |
| A | 90 | 10 | 99 |
| A | 99 | — | 99 |
| B | 90 | 10 | 99 |
| B | 99 | — | 99 |

*A 10% milk challenge was achieved by adding 99 ml of test formulation to 10 ml of sterile milk mixing and removing 1 ml for a final volume of 99 ml.

Inoculation was performed at ambient temperature with an exposure time of 30 seconds. 1% sodium thiosulfate was added to deactivate the antimicrobial components (i.e., fatty acid and iodine) of the teat dip. The inoculums were then cultured on tryptone glucose extract agar at 37° C. for 48 hours. The results of innocolum without milk challenge are shown in Table 4 and the results with milk challenge are shown in Table 5.

TABLE 4
Staphylococcus aureus ATCC 6538 Without Milk Challenge

| | | Average | | |
|---|---|---|---|---|
| Test | Survivors | Survivors | Log | Percent |
| Substance | (CFU/ml) | (CFU/ml) | Reduction | Reduction |
| A | <10 | <10 | >6.87 | >99.999 |
| A | <10 | <10 | >6.87 | >99.999 |
| B | <10 | <10 | >6.87 | >99.999 |
| B | <10 | <10 | >6.87 | >99.999 |

TABLE 5
Staphylococcus aureus ATCC 6538 With 10% Milk Challenge

| | | Average | | |
|---|---|---|---|---|
| | Survivors | Survivors | | Percent |
| Test Substance | (CFU/ml) | (CFU/ml) | Log Reduction | Reduction |
| A | 22 × 10³ | 7.8 × 10⁴ | 2.98 | 99.894 |
| A | 133 × 10³ | 7.8 × 10⁴ | 2.98 | 99.894 |
| B | <10 | <10 | >6.87 | >99.999 |
| B | <10 | <10 | >6.87 | >99.999 |

### Example 4

### Sanitizing Efficacy of Teat Dip Formulas Lacking Carboxylic Acid, Against S. aureus With and Without a 10% Milk Challenge

A teat dip formulation, Formulation N, was prepared identical to formulas A and B of Example 1, but without the carboxylic acid (heptanoic acid). The omitted 110 carboxylic acid was replaced with deionized water to provide the formulation shown in Table 6 below.

TABLE 6
Teat Dip Formulation N (No Carboxylic Acid)

| | Wt % |
|---|---|
| Deionized Water | 74.50 |
| KOH 45% | 1.00 |
| Kelzan T | 0.80 |
| Glycerine 96% | 5.50 |
| Propylene Glycol | 6.00 |
| Citric Acid | 1.90 |
| NPE 12 | 6.00 |
| Pluronic ™ 105 | 3.00 |
| NaI/I₂ Premix | 1.80 |
| | 100.00 |

1.0 ml of a Staphylococcus aureus culture having about 6.0×10⁷ cfu/ml of S. aureus was mixed with Formulation N with and without sterile milk as shown in Table 7.

TABLE 7
Test Formulas and Volumes

| | ml of Teat Dip | | Final Volume |
|---|---|---|---|
| Teat Dip Formulation | Formulation | ml of Milk | in ml* |
| N | 90 | 10 | 99 |
| N | 99 | — | 99 |

The results of bactericidal effect without milk challenge are shown in Table 8 and with milk challenge are shown in Table 9.

TABLE 8
Staphylococcus aureus ATCC 6538 Without Milk Challenge

| | | Average | | |
|---|---|---|---|---|
| | Survivors | Survivors | | Percent |
| Test Substance | (CFU/ml) | (CFU/ml) | Log Reduction | Reduction |
| N | 89 × 10¹ | 2.1 × 10³ | 4.46 | 99.996 |
| N | 336 × 10₁ | 2.1 × 10³ | 4.46 | 99.996 |

TABLE 9
Staphylococcus aureus ATCC 6538 With 10% Milk Challenge

| | | Average | | |
|---|---|---|---|---|
| | Survivors | Survivors | | Percent |
| Test Substance | (CFU/ml) | (CFU/ml) | Log Reduction | Reduction |
| N | 32 × 10⁵ | 9.7 × 10⁶ | 0.79 | 83.833 |
| N | 162 × 10⁵ | 9.7 × 10⁶ | 0.79 | 83.833 |

As will be appreciated from this Example, the lack of carboxylic acid reduces the bactericidal efficacy of the teat dip formulation.

### Example 5

### Preparation of NaI/I₂ Premix

The preparation method disclosed herein is described for a weight percentage totaling 100 weight percent of NaI/I₂ premix. 5.93 weight percent of deionized water was added to a reactor followed by 13.86 weight percent of 50% liquid NaOH with agitation. Addition of the NaOH caused a temperature increase of 7.2-10C. The reaction mixture was cooled to 29-32° C. 79.58 weight percent of coarse iodine was added to the reactor with agitation.

The mixture was then heated to 43-66° C. using tempered water. Agitation was increased and 4.56 weight percent of formic acid (90% Tech) added. The formic acid was added at a rate which maintained a temperature of about 43-52° C. Cooling was required during addition of formic acid. After addition of the formic acid, agitation was continued at 43-52° C. for about one hour. The reactor contents were then cooled to below 38° C. Additional formic acid may be added to adjust pH to about pH 4-6.5.

### Example 6

### Formulation of a Teat Dip Concentrate

Two teat dip concentrate compositions, formulations O and P can be prepared according to the methods described in Example 1 and having a formulation as set forth below in Table 10.

TABLE 10
Teat Dip Concentrate Formulations

| Formula | O | P |
|---|---|---|
| Dionized Water | 41.80 | 12.70 |
| KOH 45% | 2.00 | 3.00 |
| Kelzan T | 0.60 | 0.90 |
| Glycerine 96% | 11.00 | 16.50 |
| Propylene Glycol | 12.00 | 18.00 |
| Heptanoic Acid | 2.00 | 3.00 |
| Citric Acid | 5.00 | 7.50 |
| NPE 12 | 12.00 | 18.00 |
| Pluronic ™ P-105 | 10.00 | 15.00 |
| NaI/I₂ Premix | 3.60 | 5.40 |
| | 100.00 | 100.00 |

The formulas can be adjusted with 75% phosphoric acid to provide a working composition pH range of about 4.0 to 4.5. To prepare a working composition from formula O, one part formula O can be mixed with two parts potable water until homogeneous. To prepare a working composition from formula P, one part formula P can be mixed with three parts potable water and until homogeneous.

From the foregoing detailed description and examples, it will be evident that modifications and variations can be made to the compositions and methods of the invention without departing from the spirit and scope of the invention. Therefore, it is intended that all modifications made to the invention without departing from the spirit and scope of the invention come within the scope of the appended claims.

What is claimed is:

1. An antimicrobial composition comprising:
an iodine compound;
a C₆-C₁₂ fatty acid; and
a carrier.

2. The composition according to claim 1 wherein the iodine compound is NaI/I₂.

3. The composition according to claim 1 wherein the iodine compound is an iodophor.

4. The composition according to claim 1 wherein the fatty acid is C₇-C₉.

5. The composition according to claim 4 wherein the fatty acid is heptanoic acid.

6. The composition according to claim 1 wherein the carrier is an aqueous carrier.

7. The composition according to claim 5 wherein the composition includes at least one surfactant, the iodine compound is NaI/I₂ and a working composition provides a ratio of total surfactant:titratable I₂ of about 4:1 to 13:1.

8. The composition according to claim 7 wherein the ratio of total surfactant:titratable I₂ is about 11:1.

9. The composition according to claim 1 wherein the composition is a teat dip composition.

10. The composition according to claim 9 further comprising a rheology modifier, a surfactant and an emollient.

11. The composition according to claim 10 wherein the rheology modifier is xanthan gum.

12. The composition according to claim 10 wherein the surfactant is present in an amount greater than 5% of the total weight of the working composition.

13. The composition according to claim 12 wherein the composition is a working composition which has a ratio of total surfactant:titratable I₂ of about 4:1 to 13:1.

14. The composition according to claim 13 wherein the ratio of total surfactant:titratable I₂ is about 10:1.

15. The composition according to claim 10 further comprising glycerine.

16. The composition according to claim 7 wherein the composition is a concentrate which when diluted to a working composition provides a ratio of total surfactant:titratable I₂ of about 4:1 to 13:1.

17. A method for controlling mastitis in milk producing animals comprising:
applying an antimicrobial composition to a teat of the animal wherein the antimicrobial composition comprises an iodine compound, a C₆-C₁₂ fatty acid and a carrier.

18. The method according to claim 17 wherein the iodine compound is an iodophor.

19. The method according to claim 17 wherein the fatty acid is a C₇-C₉ fatty acid.

20. The method according to claim 19 wherein the fatty acid is heptanoic acid.

21. The method according to claim 20 wherein the pH of the composition is about pH 3.5 to 6.0.

22. The method according to claim 20 wherein the heptanoic acid is present in an amount of about 0.5 to 1.5 weight percent of a working composition.

23. The method according to claim 17 wherein the composition is a teat dip composition which forms a soft barrier.

24. An antimicrobial composition comprising
an iodine compound having a titratable iodine;
a fatty acid; and
a carrier
wherein a working composition of the antimicrobial composition provides a ratio of fatty acid:titratable iodine of about 1:25 to 3.3:1.

25. The antimicrobial composition according to claim 24 further comprising at least one surfactant wherein a working composition of the antimicrobial composition provides a ratio of total surfactant:titratable I₂ of about 4:1 to 13:1.
